(19) United States
(12) Patent Application Publication
(10) Pub. No.: US 2010/0331610 A1
(43) Pub. Date: Dec. 30, 2010

(54) Artificial insemination device for animals

(76) Inventor: Sheng-Jui Chen, Pyng-Jenn City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/457,979

(22) Filed: Jun. 26, 2009

(51) Int. Cl. A61B 17/43

(52) U.S. Cl. 600/35

(57) ABSTRACT

An improved artificial insemination device for animals is disclosed. The device has an inner catheter tube inserted inside a catheter tube where the front end of the inner catheter tube is closed in arc shape and its periphery is arranged with a plurality of spray release openings with arc-shaped invagination at outer edges and an extension member that communicates with the interior. As such, when the front end of the inner catheter tube enters the uterus along the cervical tract, it will not scratch the walls of cervical tract during movement because the outer edges of the spray release openings are in arc invaginated shape. When the contraction of the uterus sucks out the animal semen inside the inner catheter tube, it works like a straw through the extension members of the spray release openings, and the semen pooled together inside the catheter could be released rapidly and smoothly into different parts of the uterus to fully enhance the efficiency and the success rate of artificial insemination.

### BACKGROUND OF THE INVENTION

1) Field of the Invention

The present invention relates to an improved artificial insemination device, more particularly, a simple, easy to use, and convenient artificial insemination device that can enhance the safety of artificial insemination inside the animal body and improve the efficiency and success rate of artificial insemination. The invention herein is practical, ideal, and inventive and offers improvement never seen before.

2) Description of the Prior Art

Conventional artificial insemination device for animals having a catheter tube and an inner catheter tube generally have the following assemblies. As shown in FIG. 1, the catheter tube 10 is installed with a first nozzle 10 made of soft material at the front end and inserted with an inner catheter tube 20 therein. A second nozzle 21 made of soft material is installed on the front end of the inner catheter tube 20. The inner catheter tube 20 moves freely in the catheter tube 10 and the first nozzle 11. The second nozzle 21 is smaller than the first nozzle 11 and has a spray orifice 211 that is open to the exterior. To make it convenient for the fabrication and installation of the soft second nozzle 21, the second nozzle 21 is designed with a diameter bigger than the outer diameter of the inner catheter tube 20 such that the second nozzle 21 can wrap around and be secured at the front end of the inner catheter tube 20 and situated outside the first nozzle 21. As shown in FIG. 2, when artificial insemination is performed, the catheter tube 10 and the first nozzle 11 are inserted into the vaginal orifice of female animal body and carry along with them the second nozzle 21 to enter the vagina and stay at the first cervical ring area. Subsequently, the inner catheter tube 20 is pushed forward from the rear end of the catheter tube 10 to a predetermined length d, and then enters the uterus along the cervical tract along with the second nozzle 21. A stopper plug 30 is inserted between the rear end of the catheter tube 10 and the inner catheter tube 20 for positioning. Next, a flexible tube 40 is assembled between the rear end of inner catheter tube 20 and the animal semen dispenser 50 and squeezes the semen into the inner catheter tube 20. Through the contraction of the uterus, the semen squeezed into the inner catheter tube 20 is drawn out and released into the uterus from the spray orifice 211 of the first nozzle 21. Such artificial insemination device is able to achieve the purpose of artificial insemination. However there are a few drawbacks. That is, the outer diameter of the second nozzle 21 is apparently larger than the diameter of inner catheter tube 20. Thus when the second nozzle 21 enters the uterus along the cervical tract of female animal body, it would cause the animal discomfort such that the animal might be disturbed and become fidgeted, which adds to the risk of artificial insemination. Also, the flexible tube 40 installed at the rear end of inner catheter tube 20 can only be connected to a semen dispenser or an injection syringe disposed with a rigid projection 201 and cannot work with any semen bag made entirely of soft material and without a rigid projection. Its application is hence limited. Moreover, the fabrication of the second nozzle 21 requires another casting mold and separate assembly, which adds to the cost and makes the implementation less convenient.

To address the aforesaid drawbacks, some operators would use another kind of artificial insemination device as shown in FIG. 3, FIG. 4 and FIG. 5. The second nozzle 21 of such insemination device has an outer diameter roughly identical to that of the inner catheter tube 20 and has through spray orifices 211 at the periphery so as to improve the discomfort experienced by the animal during the artificial insemination procedure. However to render the circumference of the second nozzle 21 equidiameter to the inner catheter tube 20 (the outer diameter of inner catheter is typically φ3 mm), the second nozzle 21 must be disposed with a smaller-diameter connecting member 212 at its rear end that can be inserted into the inner diameter of the inner catheter tube 20 such that the center orifice 2121 of the connecting member 212 is far smaller than the inner diameter of the inner catheter tube 20. As such, when artificial insemination is performed, the semen in the inner catheter tube 20 will have smaller flow when it passes through the center orifice 2121, rendering the insemination process less smooth and more time consuming. Also, in order to assemble the second nozzle 21 to the inner catheter tube 20, the second nozzle 21 must be made of rigid plastic material by injection molding. As such, the edges 2111 around the circumference of the spray orifice 211 of the resulting second nozzle 21 are likely to have burrs or sharpness, and its junction A with the inner catheter tube 20 tends to have gap, step or burr. When the second nozzle 21 enters the uterus along cervical tract during artificial insemination, the soft walls of the cervical tract are vulnerable to scratches by burrs, assembly gap and step, which may even lead to inflammation or bacterial infection. And again, the fabrication of the second nozzle 21 requires another casting mold and separate assembly, which similarly adds to the cost and makes the implementation less convenient. Moreover, as shown in FIG. 6, a flexible tube 40 at the rear end of the inner catheter tube 20 can only be connected to a semen dispenser 50 with a projection 501 and cannot be assembled to a semen dispenser made of soft material.

### SUMMARY OF THE INVENTION

The primary object of the present invention is to provide an improved artificial insemination device for animals, characterized in which when the front end of the inner catheter tube extends inwardly into the uterus of the animal, it will not cause any discomfort or unsafeness and will enable the semen to be pooled together and released rapidly and smoothly into different parts of the uterus, thereby enhancing the efficiency and success rate of artificial insemination.

Another object of the present invention is to provide an improved artificial insemination device that can be used with both semen dispenser or injection syringe having a rigid projection, and semen bag made entirely of soft material, hence expanding its scope of application.

To achieve the aforementioned objects, the improved artificial insemination device for animals provided by the present invention is characterized by having an inner catheter tube where the front end of the inner catheter tube is closed in arc shape and its periphery is arranged with a plurality of spray release openings with arc-shaped invagination at outer edges and having an extension member communicating with the interior, and the rear end of the inner catheter tube is disposed with a connector. As such, when the front end of the inner catheter enters the uterus along the cervical tract, it will not scratch the walls of cervical tract during movement because the outer edges of the spray release openings are in arc invaginated shape. When the contraction of the uterus sucks out the animal semen inside the inner catheter, it works like a straw through the extension members of the spray release openings, and the semen pooled together inside the catheter could be released rapidly and smoothly into different parts of the uterus to fully enhance the efficiency and the success rate of artificial insemination. In addition, because the connector installed at the rear end of the inner catheter tube is made of rigid material, it can be connected to the sheath member of the projection of a semen dispenser or an injection syringe or serves as an insertion member for connecting a semen bag, hence giving the artificial insemination device broader application.

The objects, features and effects of the invention are described in details below with accompanying drawing and embodiments.

### BRIEF DESCRIPTION OF THE DRAWINGS

FIG. 1 is a schematic view of a conventional artificial insemination device for animals.

FIG. 2 is a schematic view of the artificial insemination device in FIG. 1 in operation.

FIG. 3 is a schematic view of another conventional artificial insemination device for animals.

FIG. 4 is a prospective view of a second nozzle of the conventional artificial insemination device shown in FIG. 3.

FIG. 5 is an assembly view of an inner catheter tube and a second nozzle of the conventional artificial insemination device shown in FIG. 3.

FIG. 6 is a schematic view of the artificial insemination device in FIG. 3 in operation.

FIG. 7 is a schematic view of an embodiment of the invention.

FIG. 8 is a prospective view of the front end of an inner catheter in the embodiment of the invention.

FIG. 9 is a schematic view of FIG. 8.

FIG. 10 is a schematic view of the embodiment operating with a semen dispenser.

FIG. 11 is a schematic view of the embodiment operating with a semen bag.

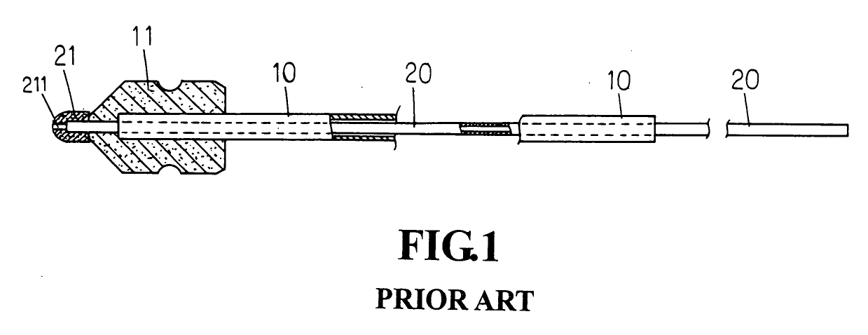

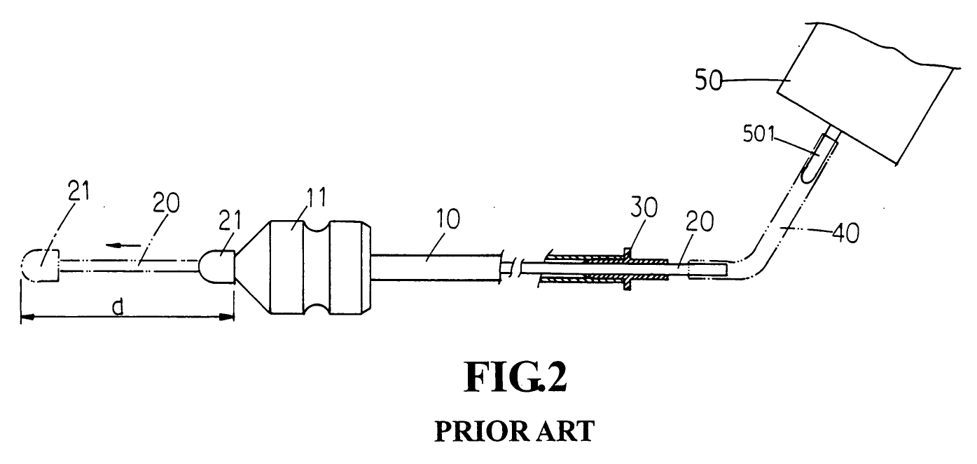

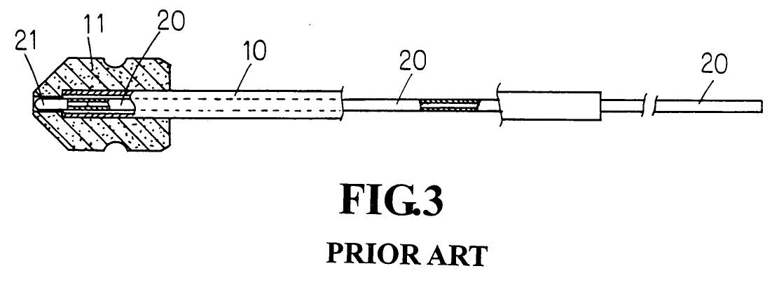

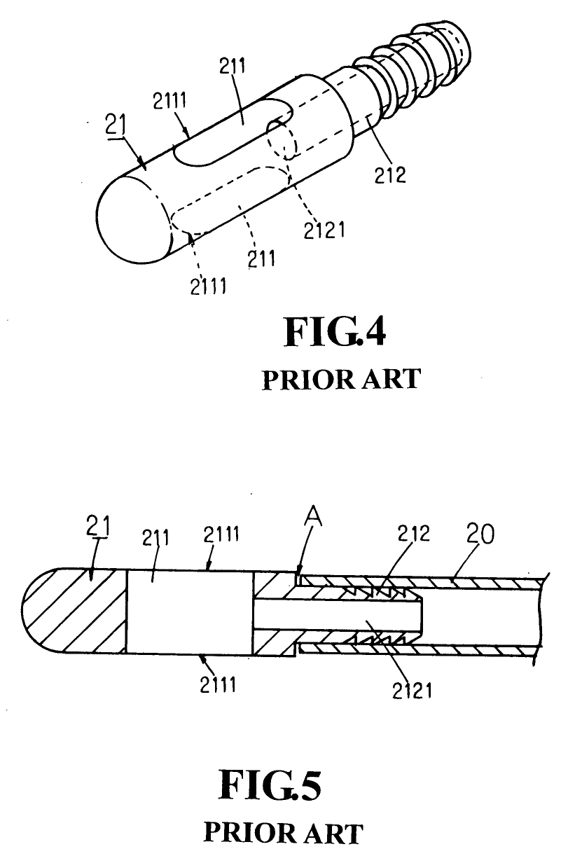

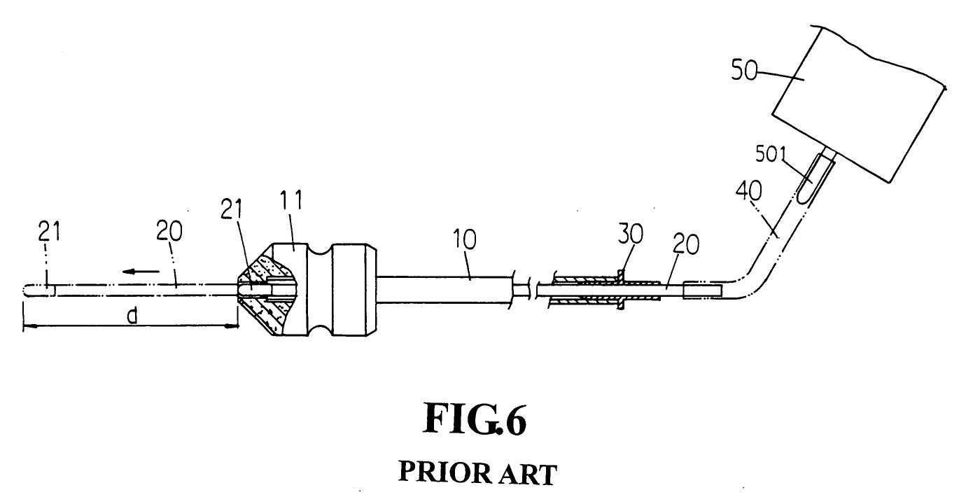

### DETAILED DESCRIPTION OF THE INVENTION

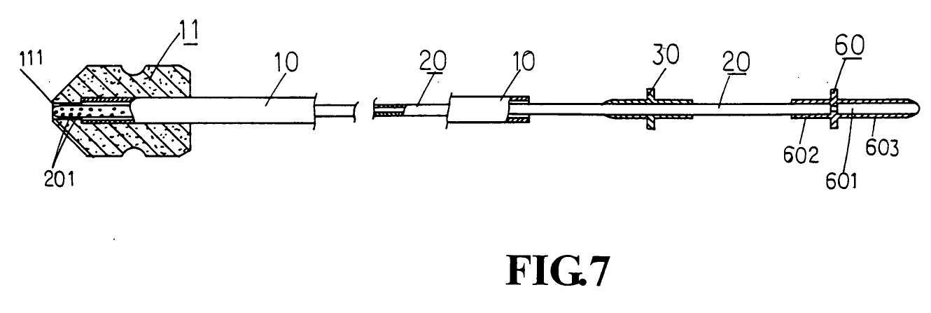

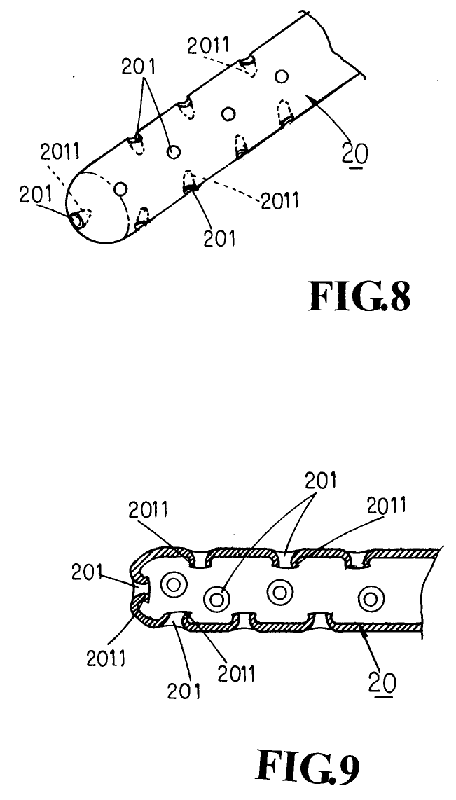

As show in FIG. 7, FIG. 8 and FIG. 9, the improved artificial insemination device for animals according to the invention features a catheter tube 10, the catheter tube 10 being a flexible plastic tube and having a first nozzle 11 made of soft material arranged at the front end, the first nozzle 11 having a guide orifice 111 at the center communicating with the exterior; an inner catheter tube 20, the inner catheter tube 20 being a flexible plastic tube freely disposed inside the catheter tube 10 and its front end being closed in arc shape and its periphery being arranged with a plurality of spray release openings 201 with arc-shaped invagination at outer edges and having an extension member 2011 communicating with the interior; a stopper plug 30 passing through the adjoining of rear end of the inner catheter tube 20 and being plugged into the rear end of catheter tube 10 for positioning the inner catheter tube 20; and a connector 60, the connector 60 being made of flexible material with proper rigidity and having a through hole 601 at the center, its one end 602 being sleeved over the rear end of the inner catheter tube 10, and its other end 603 being for sleeving over the projection of an animal semen dispenser or an injection syringe, or for insertion into a slot on a soft semen bag.

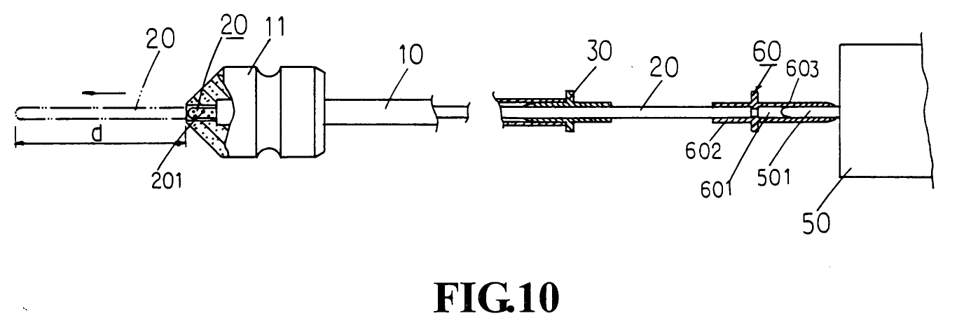

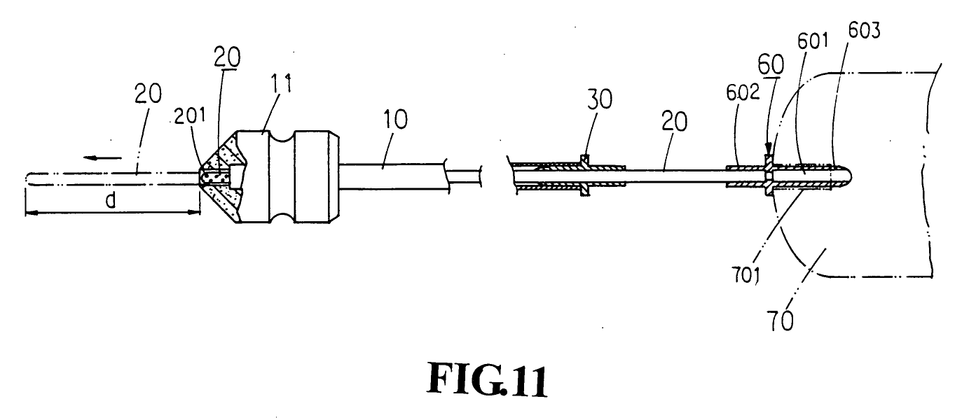

As shown in FIG. 10, when performing artificial insemination, the catheter tube 10 of the invention and the inner catheter tube 20 disposed therein are extended inwardly through the vaginal orifice of the female animal body, and the first nozzle 11 is positioned at the location of the first cervical ring. Next, push the inner catheter tube 20 forward for a predetermined length d such that its front end enters the uterus, then plug the stopper plug 30 between the rear end of the catheter tube 10 and the inner catheter tube 20 for positioning. Next, insert the projection 501 of the semen dispenser 50 into the connector 60 installed at the rear end of the inner catheter tube 20, and squeeze the semen inside the semen dispenser into the inner catheter tube 20. Through the contraction of the uterus, animal semen contained inside the inner catheter tube 20 would be sucked out through the spray release openings 201. Because each spray release opening 201 has an extension member 2011 that extends inside the tube, it produces a straw action to draw out semen. The semen inside the tube can then be pooled together by the extension members 2011 and released rapidly and smoothly into different parts of the uterine, including the uterine horns, thereby enhancing the efficiency and success rate of artificial insemination. In addition, as the front end and the body of the inner catheter tube 20 are in one piece, its fabrication is made simple, convenient, fast and economical. In particular, because the outer edges of the spray release openings are in arc invaginated shape and free of sharp burrs or steps, when the front end of the inner catheter tube 20 moves inwardly inside the uterus along the cervical tract, the soft walls of the cervical tract will not be scratched by the moving spray release orifices to ensure the safety of the female animal.

Again referring to FIG. 11, because the connector 60 arranged at the rear end of inner catheter tube 20 has proper rigidity, it can be connected to a semen dispenser or an injection syringe 50 having a projection 501 (as shown in FIG. 10). When a semen bag made entirely of soft material and not having a projection is used, the connector 60 can be inserted directly into the preconfigured slot 701 on the semen bag 70 to draw out the semen. Thus the present invention has broader application.

To sum up, the present invention offers at least the following advantages:
1. Its fabrication is simple, convenient, quick and economical.
2. It enhances the efficiency and the success rate of artificial insemination.
3. It enhances the safety of artificial insemination.
4. It has extended applications to semen dispenser, injection syringe and semen bag with or without a projection.

1. An improved artificial insemination device for animals, comprising a catheter tube, the catheter tube being a flexible plastic tube arranged with a soft first nozzle at the front end and the first nozzle having a guide orifice at the center communicating with the exterior; an inner catheter tube, the inner catheter tube being a flexible plastic tube disposed freely inside the catheter tube; and a stopper plug passing through adjoining rear end ( [symbol] ) of the inner catheter tube and being plugged into the rear end of the catheter tube for positioning; characterized in which the front end of the inner catheter is closed in arc shape and its periphery is arranged with a plurality of spray release openings with arc-shaped invagination at outer edges and having an extension member communicating with the interior.

2. The improved artificial insemination device for animals according to claim 1, wherein a connector is installed at the rear end of the inner catheter tube.

3. The improved artificial insemination device for animals according to claim 2, wherein the connector is made of flexible material with proper rigidity and has a through hole at the center, its one end being sleeved over the rear end of the inner catheter tube, and its other end being for sleeving over the projection of an animal semen dispenser or an injection syringe, or for insertion into a slot on a soft semen bag.
